(19) United States
(12) Patent Application Publication
(10) Pub. No.: US 2018/0327453 A1
(43) Pub. Date: Nov. 15, 2018

(54) HER-2-SPECIFIC CYCLIZED SUPR PEPTIDES

(71) Applicants: Stephen FIACCO, South Pasadena, CA (US); Amanda HARDY, Pasadena, CA (US); EVORX TECHNOLOGIES, INC., Pasadena, CA (US)

(72) Inventors: Stephen FIACCO, South Pasadena, CA (US); Amanda HARDY, Pasadena, CA (US)

(73) Assignee: EVORX TECHNOLOGIES, INC., Pasadena, CA (US)

(21) Appl. No.: 15/774,141

(22) PCT Filed: Nov. 7, 2016
(86) PCT No.: PCT/US2016/000107

§ 371 Date: May 7, 2018

Related U.S. Application Data
(60) Provisional application No. 62/251,824, filed on Nov. 6, 2015.

(51) Int. Cl. C07K 7/06; C07K 7/56; A61K 38/00

(52) U.S. Cl. CPC C07K 7/06; A61K 38/00; C07K 7/56

(57) ABSTRACT

Disclosed are highly potent peptides, peptide conjugates, host cells and compositions containing one or more of these peptides, peptide conjugates and/or host cells that are useful to inhibit the growth of a breast cancer cell or treat breast cancer in a patient in need of such treatment.

### CROSS REFERENCE TO RELATED APPLICATIONS

This application is a U.S. National Stage Application of International Application No. PCT/US2016/000107 filed Nov. 7, 2016, which claims the benefit of the filing date of U.S. Provisional Application No. 62/251,824, filed Nov. 6, 2015. The entirety of all the above-listed applications are incorporated herein by reference.

### BACKGROUND

HER-2 (Human epithelial growth factor 2) is a receptor tyrosine kinase having no known ligand, It is expressed in 20% to 30% of breast cancer patients (Lin et al. (2007) Clin. Canc. Res. 13:1648), with over 50,000 cases being diagnosed with it yearly, and many being lethal. The overexpression of HER-2 in breast cancer correlates to a more invasive disease with increased tumor growth, chemotherapy resistance, and significantly lower long term survival for patients (Sausville et al. (2006) Canc. Res. 66:3351). This is because Her2, when dimerized with other Her family member (1, 3, and 4), promotes the stimulation of cellular proliferation, invasion, and anti-apoptosis (Lin et al. (2007) supra).

Monoclonal antibody therapies exist for Her 2(+) breast cancer. Examples include Herceptin (Trastuzumab), which binds to the extracellular domain of Her 2, and Pertuzumab (Perj eta) which inhibits the dimerization of Her 2 with other Her 2 recpetors. Trastuzumab emtansine (T-DM1) is an antibody—drug conjugate incorporating the Her 2—targeted antitumor properties of trastuzumab with the cytotoxic activity of the microtubule-inhibitory agent DM1. However, antibody therapeutics are expensive to develop, are not orally available, are painful upon administration via large IV injections, and their size limits their efficacy with solid tumors (Cho et al. (1996) Trends in Biotechnol. 14:153).

There are also small molecule therapies including Lapatinib (INN), used in the form of lapatinib ditosylate, (USAN) (Tykerb/Tyverb, GSK) which is an orally active drug for breast cancer and other solid tumours. It is a dual tyrosine kinase inhibitor which interrupts the HER2/neu and epidermal growth factor receptor (EGFR) pathways (Higa et al. (2007) Expert Rev. Antican. Ther. (Future Drugs) 7(9):1183-92). However, significant side effects including diarrhea, fatigue, nausea, rashes, and elevated liver enzymes and QT-prolongation have been reported with the use of this drug.

Thus, what is needed are better therapeutics with fewer side effects for treating HER-2(+) breast cancer.

### SUMMARY

It has been discovered that Scanning Unnatural Protease Resistant (SUPR) cyclized peptides are able to target themselves to in vivo HER-2+ breast cancer primary tumors and metastatic cancers, enabling imaging and treatment. This discovery has been exploited to provide specifically cyclized SUPR peptides and methods of making and using the same.

In one aspect, the disclosure provides a non-naturally occurring peptide comprising an amino acid sequence (MVCVVLYDDK) (SEQ ID NO:1), wherein Position 1 is Met or (norvaline, norleucine, alanine); Position 2 is Val or (I, L); Position 3 is Cys, Position 4 is N-methyl norvaline or (Y, F, P, D, E, M); Position 5 is N-methyl norvaline or (Y, F, D, E, W, C, G, P); Position 6 is Leu or (Y, F, V, V, I, P, C); Position 7 is Tyr or (V, E, D); Position 8 is Asp or (S, T, E, Y, F, A, P, V); Position 9 is Asp or (E, G, L, I, V); and Position 10 is Lys or (lysine derivatives e.g., Orn).

In some embodiments, the non-naturally occurring peptide is cyclized.

In some embodiments, the cyclized SUPR peptide of claim 1 has the structure (FIG. 1), (FIG. 2), (FIG. 3A), (FIG. 3B), (FIG. 3C), (FIG. 4A), (FIG. 4B), (FIG. 4C), (FIG. 5A), (FIG. 5B), (FIG. 5C), (FIG. 5D), (FIG. 5E), (FIG. 5F), or (FIG. 6). In other embodiments, the SUPR is linear and has the structure (FIG. 7A) or (FIG. 7B).

In some embodiments, the invention provides a non-naturally occurring peptide of claim 1, comprising a label, which in certain embodiments is a dye, such as, but not limited to, Cy5.

In other embodiments, the label is an in vivo imaging agent such as, but not limited to, 18F. In further embodiments, the label is a cytotoxin or radioactive atom, such as 90Y, that can be used for therapeutic applications.

The SUPR peptide of claim 1 may also comprise a pharmaceutically acceptable carrier.

In another aspect, the disclosure provides a method for inhibiting the growth of an HER-2+ breast cancer cell, comprising contacting the cell with a therapeutically effective amount of the cyclized SUPR peptide as described above, and a method for treating HER-2+ breast cancer in subject in need thereof, comprising administering to the subject the therapeutically effective amount of the cyclized SUPR peptide as described above.

Also provided is a method for producing non-naturally occurring peptides.

A method imaging a HER-2+ cell in a subject is also provided in another aspect. This method comprises administering to the subject the a labelled HER-2-specific cyclized SUPR peptide as described above in an amount sufficient to image the cell.

In another aspect, the disclosure provides a kit for one or more of: inhibiting the growth of a HER-2+ breast cancer cell, for treating a HER-2+ breast cancer, or for imaging a HER-2+ cancer in a subject. The kit comprises the non-naturally occurring peptide of claim 1 and instructions for use. In one embodiment, the kit further comprises a label.

### BRIEF DESCRIPTIONS OF THE FIGURES

The foregoing and other objects of the present disclosure, the various features thereof, as well as the invention itself may be more fully understood from the following description, when read together with the accompanying drawings in which:

FIG. 1 is a schematic representation of a cyclized SUPR peptide according to the disclosure, where the side chain of Lys is linked via a linker to the N-terminus (C terminus has been amidated);

FIG. 2 is a schematic representation of a side chain to side chain cyclized SUPR peptide according to the disclosure, where an N-terminal Lys has been added, and the side chain of Lys is linked via a linker to the side chain of the N-terminal Lys;

FIG. 3A is a schematic representation of a side chain to N-terminus cyclized SUPR peptide according to the disclosure, where the side chain of Lys derivative Dap is linked via a linker to the N-terminus (Glutarate to C-terminal Dap);

FIG. 3B is a schematic representation of a side chain to N-terminus cyclized SUPR peptide according to the disclosure, where the side chain of Lys derivative Dab is linked via a linker to the N-terminus (Glutarate to C-terminal Dab);

FIG. 3C is a schematic representation of a side chain to N-terminus cyclized SUPR peptide according to the disclosure, where the side chain of Lys derivative Orn is linked via a linker to the N-terminus (Glutarate to C-terminal Orn);

FIG. 4A is a schematic representation of a side chain to side chain cyclized SUPR peptide according to the disclosure, where an N-terminal Asp was added to the sequence and linked to C-terminus of Orn;

FIG. 4B is a schematic representation of a side chain to side chain cyclized SUPR peptide according to the disclosure, where an N-terminal Asp was added to the sequence and linked to C-terminus of Lys;

FIG. 4C is a schematic representation of a side chain to side chain cyclized SUPR peptide according to the disclosure, where an N-terminal Glu was added to the sequence and linked to C-terminus of Lys;

FIG. 5A is a schematic representation of a cyclized SUPR peptide according to the disclosure, where a side chain of Orn is linked to the C-terminus;

FIG. 5B is a schematic representation of a cyclized SUPR peptide according to the disclosure, where a side chain of Lys is linked to the C-terminus;

FIG. 5C is a schematic representation of a cyclized SUPR peptide according to the disclosure, where a side chain of HomoBeta-Lys is linked to the C-terminus;

FIG. 5D is a schematic representation of a cyclized SUPR peptide according to the disclosure, where a side chain of Orn is linked to the C-terminal Gly;

FIG. 5E is a schematic representation of a cyclized SUPR peptide according to the disclosure, where a side chain of Lys is linked to the C-terminal Gly;

FIG. 5F is a schematic representation of a cyclized SUPR peptide according to the disclosure, where a side chain of Lys is lined to the C-terminal Beta-Alanine;

FIG. 6 is a schematic representation of a cyclized SUPR peptide according to the disclosure, where the N-terminus is linked to the C-terminus;

FIG. 7A is a schematic representation of an SUPR peptide which is a Ser derivative having HomoSer at position 3;

FIG. 7B is a schematic representation of an SUPR peptide which is a Ser derivative having N-methyl Ser at position 3;

FIG. 8 is a graphic representation of the binding of SUPR peptide as determined by FACs using SKOV3 cells.

FIG. 9A is a representation of a fluorogram of the dorsal view of a SKOV3 mouse model injected with 50 nM SUPR-4m-Cy5 after 3 hours;

FIG. 9B is a representation of a fluorogram of the ventral view of a SKOV3 mouse model injected with 50 nM SUPR-4m-Cy5 after 3 hours;

FIG. 9C is a representation of a fluorogram of the major organ systems removed from a SKOV3 mouse model that had been injected with 50 nM SUPR-4m-Cy5 after 3 hours;

FIG. 10A is a representation of a fluorogram of SKOV3 (HER-2+) mice and MDA-MB-231 (HER-2 negative) mice, treated with 10 nM SUPR-4m-Cy5, and of a SKVO3 control mouse treated with Pertumab, and each imaged after 2 hours;

FIG. 10B is a representation of a fluorogram of SKOV3 (HER-2+) mice and MDA-MB-231 (HER-2 negative) mice, treated with 10 nM SUPR-4m-Cy5, and of a SKVO3 control mouse treated with Pertumab, and each imaged after 4 hours; and

FIG. 10C is a representation of a fluorogram of SKOV3 (HER-2+) mice and MDA-MB-231 (HER-2 negative) mice, treated with 10 nM SUPR-4m-Cy5, and of a SKVO3 control mouse treated with Pertumab, and each imaged after 6 hours.

### DETAILED DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

The present disclosure described the use of Scanning Unnatural Protease Resistant (SUPR) peptides as in vivo imaging agents for HER-2 (+) breast cancer. By utilizing PET-labeled SUPR peptides that recognize HER-2, information about the primary tumor as well as metastases can be obtained without the need for biopsy.

In particular, this disclosure provides non-naturally occurring cyclized and linear SUPR peptide comprising modifications having the following peptide sequences: MVCVVLYDDK (SEQ ID NO:1), wherein Position 1 is Met or (norvaline, norleucine, alanine); Position 2 is Val or (I, L); Position 3 is Cys, Position 4 is N-methyl norvaline or (Y, F, P, D, E, M); Position 5 is N-methyl norvaline or (Y, F, D, E, W, C, G, P); Position 6 is Leu or (Y, F, V, V, I, P, C); Position 7 is Tyr or (V, E, D); Position 8 is Asp or (S, T, E, Y, F, A, P, V); Position 9 is Asp or (E, G, L, I, V); Position 10 is Lys or (lysine derivatives e.g., Orn). Examples of N-methyl amino acid include, but are not limited to N-methyl norvaline or N-methyl alanine or alternatively any modification to an amino acid that confers stabilization, e.g., proline, D-amino acids, Beta amino acids, peptoids, and 2-aminoisobutyric acid (Aib).

The non-naturally occurring peptides of the disclosure can be modified to include unnatural amino acids. Thus, the peptides may comprise D-amino acids, a combination of D- and L-amino acids, and various “designer” amino acids (e.g., .beta.-methyl amino acids, C-.alpha.-methyl amino acids, and N-.alpha.-methyl amino acids, etc.) to convey special properties to peptides. Additionally, by assigning specific amino acids at specific coupling steps, peptides with .alpha.-helices .beta. turns, .beta. sheets, .gamma.-turns, and cyclic peptides can be generated. Generally, it is believed that .alpha.-helical secondary structure, beta sheet, and gamma turns are useful.

The SUPR peptide according to the disclosure can be obtained by chemical synthesis using a commercially available automated peptide synthesizer such as those manufactured by Perkin/Elmer/Applied Biosystems, Inc., Model 430A or 43La (Foster City, Calif., USA). The synthesized polypeptide can be precipitated and further purified, for example by high performance liquid chromatography (HPLC), and then cyclized. Accordingly, this disclosure also provides a process for chemically synthesizing the SUPR peptides of this disclosure by providing the sequence of the protein and reagents, such as amino acids and enzymes and linking together the amino acids in the proper orientation and linear sequence. Alternatively, the proteins and polypeptides can be obtained by well-known recombinant methods as described, for example, in Sambrook et al. (1989) supra, using a host cell and vector systems described herein.

In one aspect, the disclosure provides non-naturally occurring cyclized SUPR peptides derived from a peptide having the following amino acid sequence: MVCVVLYDDK (SEQ ID NO:1) (EP 2751291A1), wherein Position 1 is Met or norvaline, or norleucine, or alanine; Position 2 is Val or (I, L); Position 3 is Cys, Position 4 is N-methyl norvaline or (Y, F, P, D, E, M); Position 5 is N-methyl norvaline or (Y, F, D, E, W, C, G, P); Position 6 is Leu or (Y, F, V, V, I, P, C); Position 7 is Tyr or (V, E, D); Position 8 is Asp or (S, T, E, Y, F, A, P, V); Position 9 is Asp or (E, G, L, I, V); Position 10 is Lys or (lysine derivatives e.g., Orn), and for each of the above, V is an N-methyl amino acid or any modified amino acid that confers stabilization to the peptide. The original sequence was described in WO 2013/033626.

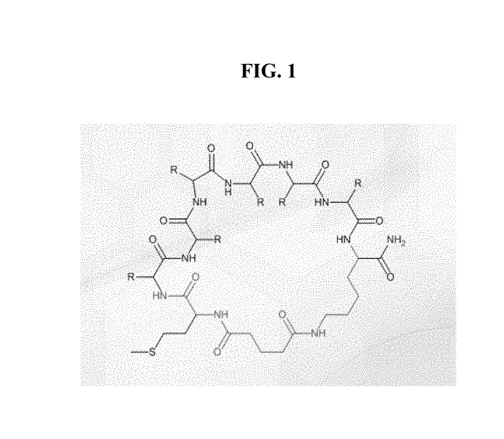

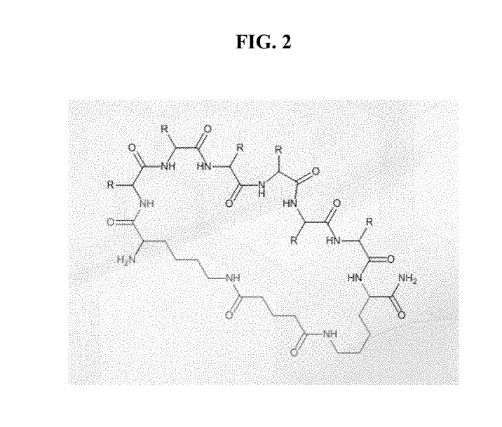

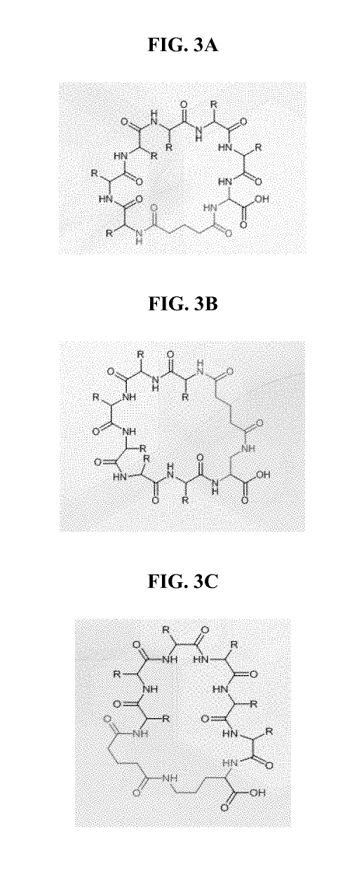

The SUPR peptides are cyclized by derivatizing the peptide of SEQ ID NO:1 in order to cyclize it. This can be accomplished in multiple ways. For example, a side chain of Lys in the peptide can be linked to the N-terminus of the peptide via a linker (FIG. 1). Alternatively, a Lys residue can be added to the N-terminus of the peptide and linked via its side chain to the side chain of another Lys in the peptide via a linker (FIG. 2). In another example, a Lys derivative such as Dap, Dab, or Orn, can be used in the peptide in place of Lys, and their side chain linked to the N-terminus of the peptide by a linker (FIGS. 3A-3C). For example, C-terminal Dap, Orn, or Dab can be linked to N-terminal Glu via a linker. The C-terminus can be amidated or not.

Useful linkers for these purposes include non-limiting examples such as succinic acid, glutaric acid, adipic acid and primelic acid.

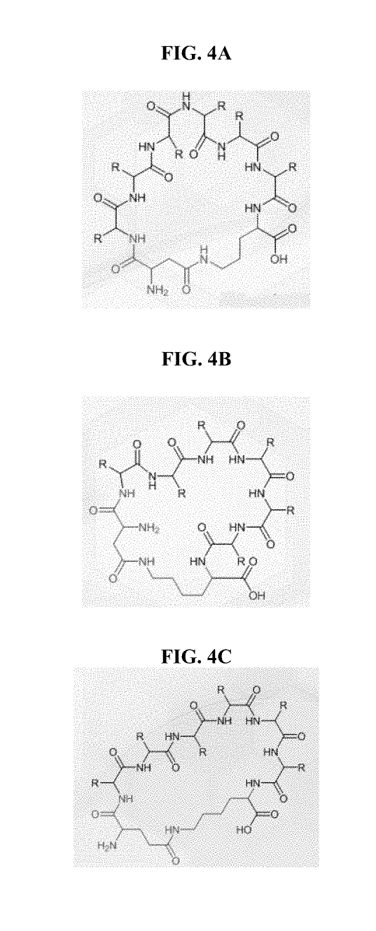

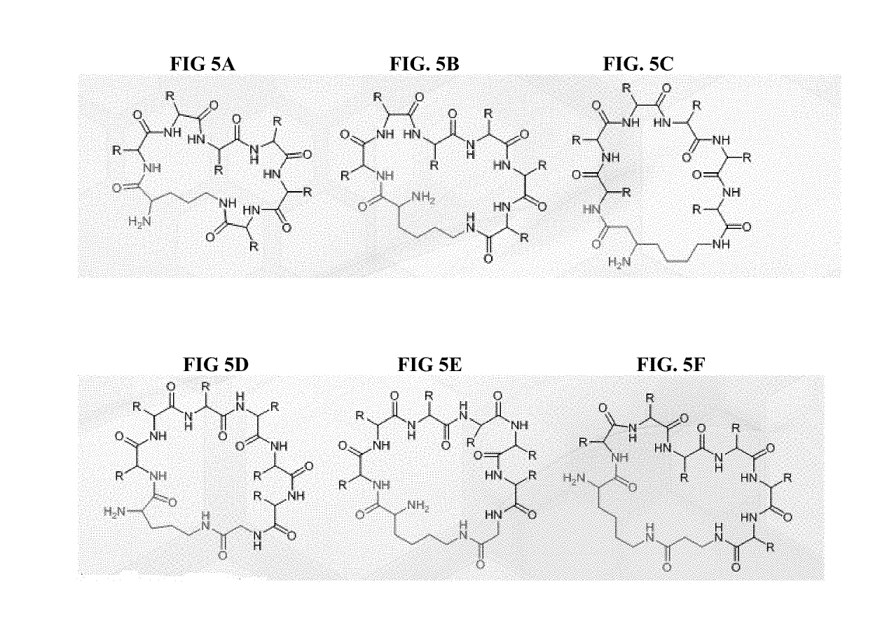

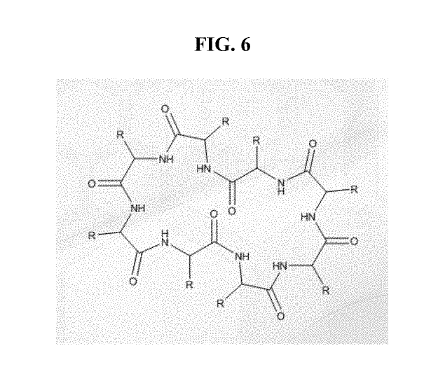

Other modes of SUPR cyclization not using linkers include residue side chain to side chain cyclization. For example, an N-terminal Asp or Glu can be added which can be linked to a C-terminal Lys or Lys derivative such as Dab, Dap, or Orn (FIGS. 4A-4C). Alternatively, the side chains of Lys or a Lys derivative or can be coupled to the C terminus, or ro a c-terminal Gly or Beta-Alanine (FIGS. 5A-5F). In yet another mode, the N-terminus of the SUPR peptide can be directly linked to its C-terminus (FIG. 6).

Non-limiting useful substitutions are serine derivatives at position 3 such as homoserine, N-methyl-Ser, Thr, N-methyl Thr, Hse, and N-methyl Hse.

The compositions are useful to inhibit the growth of a breast cancer cell in vitro or in vivo. In one aspect, the contacting is in vivo and a therapeutically effective amount of the composition is administered. In a further aspect, the patient is a HER2+ patient.

The SUPR peptides according to the disclosure may contain one or more radionuclides which are suitable for use as radio-imaging agents or as therapeutics for the treatment of rapidly proliferating cells, for example, HER-2+ expressing cancer cells. Accordingly, in one embodiment, a pharmaceutical composition is provided including a complex that includes a metal and a cyclized SUPR peptide according to the disclosure, or a salt, solvate, stereoisomer, or tautomer thereof, and a pharmaceutically acceptable carrier.

As used herein, the term “label” intends a directly or indirectly detectable compound or composition that is conjugated directly or indirectly to the composition to be detected, e.g., N-terminal histadine tags (N-His), magnetically active isotopes, e.g., 115Sn, 117Sn and 119Sn, a nonradioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described, for example, in Haugland (1996) Handbook of Fluorescent Probes and Research Chemicals (6th ed.) chapters 1 and 11-23, https://www.thermofisher.com/us/en/home/references/molecular-probes-the-handbook.html. Examples of luminescent probes include, but are not limited to, aequorin and luciferases. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red.

A pharmaceutical composition is also provided, which is suitable for in vivo imaging and radiotherapy. Suitable pharmaceutical compositions may contain a radio imaging agent, or a radiotherapeutic agent that has a radionuclide either as an element, i.e., radioactive iodine, or a radioactive metal chelate complex of the SUPR peptides in an amount sufficient for imaging, together with a pharmaceutically acceptable radiological vehicle. The radiological vehicle are suitable for injection or aspiration, such as human serum albumin; aqueous buffer solutions, e.g., tris(hydromethyl)aminomethane (and its salts), phosphate, citrate, bicarbonate, etc.; sterile water; physiological saline; and balanced ionic solutions containing chloride and or dicarbonate salts or normal blood plasma cations such as calcium, potassium, sodium, and magnesium.

The concentration of the imaging agent or the therapeutic agent in the radiological vehicle should be sufficient to provide satisfactory imaging. For example, when using an aqueous solution, the dosage is about 1.0 mCi to 50 mCi. The actual dose administered to a patient for imaging or therapeutic purposes, however, is determined by the physician administering treatment. The imaging agent or therapeutic agent should be administered so as to remain in the patient for about 1 hour to 24 hours, although both longer and shorter time periods are acceptable. Therefore, convenient ampoules containing 1 mL to 10 mL of aqueous solution may be prepared.

Imaging may be carried out in the normal manner, for example by injecting a sufficient amount of the imaging composition to provide adequate imaging and then scanning with a suitable machine, such as a gamma camera. In certain embodiments, a method of imaging a region in a patient includes the steps of: (i) administering to a patient a diagnostically effective amount of a compound complexed with a radionuclide; exposing a region of the patient to radiation; and (ii) obtaining an image of the region of the patient.

In another aspect, a method of imaging a region in a patient is provided including administering to a patient a diagnostically effective amount or a therapeutically effective amount of an SUPR peptide complexed to a metal, or a pharmaceutically acceptable salt or solvate, and obtaining an image of the region of the patient. The metal used to form the complex is a radionuclide selected from ¹¹¹In, ⁹⁰Y, ⁶⁸Ga, ⁶⁴ Cu, ¹⁵³Gd, ¹⁵⁵Gd, ¹⁵⁷Gd, Fe, or ¹⁷⁷Lu.

The amount of an SUPR peptide according to the disclosure, or a formulation comprising a complex of a metal and an SUPR peptide, or its salt, solvate, stereoisomer, or tautomer that is administered to a patient depends on several physiological factors that are routinely used by the physician, including the nature of imaging to be carried out, tissue to be targeted for imaging or therapy and the body weight and medical history of the patient to be imaged or treated using a radiopharmaceutical.

Accordingly in another aspect, the invention provides a method for treating a patient for a HER-2+ cancer by administering to a patient a therapeutically effective amount of an SUPR peptide according to the disclosure complexed to a radionuclide, or a pharmaceutically acceptable salt or solvate of the complex, to treat a patient suffering from a cell proliferative disease or disorder in which HER-2+ is overexpressed. Specifically, the cell proliferative disease or disorder to be treated using a radiopharmaceutical in accordance with this invention is a breast cancer.

The present invention, thus generally described, will be understood more readily by reference to the following examples, which are provided by way of illustration and are not intended to be limiting of the present invention.

In another aspect the SUPR peptides can be combined with a carrier, such as a pharmaceutically acceptable carrier, for diagnostic and/or therapeutic use.

The cyclized and linear SUPR peptides of this disclosure also can be combined with various solid phase and pharmaceutically acceptable carriers for diagnostic and/or therapeutic use, such as an implant, a stent, a paste, a gel, a dental implant, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils.

This disclosure also provides a pharmaceutical composition comprising or alternatively consisting essentially of, or yet further consisting of, any of the cyclized SUPR peptides of this disclosure, alone or in combination with each other, or with other anti-cancer agents, and an acceptable carrier or solid support.

For oral preparations, any one or more of polypeptide as can be used alone or in pharmaceutical formulations of the disclosure comprising, or consisting essentially of, the peptide, or the peptide and other agents in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Aerosol formulations provided by the disclosure can be administered via inhalation and can be propellant or non-propellant based. For example, embodiments of the pharmaceutical formulations of the disclosure comprise a cyclized SUPR peptide of the disclosure formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself (e.g., by an elastic bladder)).

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds of the disclosure. Similarly, unit dosage forms for injection or intravenous administration may comprise a compound of the disclosure in a composition as a solution in sterile water, normal saline or another pharmaceutically acceptable carrier.

Other pharmaceutical formulations of the disclosure include those in which one or more of a cyclized SUPR peptide according to the disclosure is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

The pharmaceutical formulation may also be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

The disclosure also provides a drug delivery system for cyclized SUPR administration from which is at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a peptide of the disclosure are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985.

The composition or formulation to be administered will, in any event, contain a quantity of the compound adequate to achieve the desired state in the subject being treated.

Compositions of the present disclosure include those that comprise a sustained-release or controlled release matrix.

In general, pharmaceutical formulations containing metal complexes SUPR peptides, or pharmaceutical compositions thereof, may be administered orally, or via a parenteral route, usually by injection. Parenteral routes include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. In some embodiments, the compound, or pharmaceutical composition thereof, is administered orally. Such compositions may take the form of tablets, pills, capsules, semisolids, powders, solutions, suspensions, elixirs, aerosols, or any other appropriate compositions.

Also provided by this disclosure is a method for inhibiting the growth of a breast cancer cell, comprising, or alternatively consisting essentially of, or yet further consisting of, contacting the cell with an effective amount of a non-naturally occurring peptide, the conjugate, or a composition of this disclosure, or a combination of any thereof. Contacting can be in vitro or in vivo. When performed in vitro, the method is a useful pre-clinical screen. When the method is performed in vivo in an animal such as a mouse or other animal model, it is a secondary preclinical screen for the testing of candidate agents.

Further provided are methods for treating breast cancer in subject in need thereof, comprising consisting essentially of, or yet further consisting of, administering to the subject an effective amount of one or more of the non-naturally occurring peptide, the conjugate, or a composition of this disclosure, or a combination of any thereof. In one aspect, the subject is a human patient. In a further aspect, the human patient is HER+patient.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. The skilled artisan will be able to determine appropriate amounts depending on these and other factors.

The compositions and related methods of the present disclosure may be used in combination with the administration of other therapies. These include, but are not limited to, the administration of anticancer drugs and compositions. The additional therapeutic treatment can be added prior to, concurrent with, or subsequent to methods or compositions used to treat the cancer, and can be contained within the same formulation or as a separate formulation.

The present disclosure provides methods for screening for equivalent agents, such as equivalent peptides to a peptide or composition of this disclosure, and various agents that modulate the activity of the active agents and pharmaceutical compositions of the disclosure or the function of a polypeptide or peptide product encoded by the polynucleotide of this disclosure. For the purposes of this disclosure, an “candidate agent” is intended to include, but not be limited to a biological or chemical compound such as a simple or complex organic or inorganic molecule, a peptide, a protein (e.g., antibody), a polynucleotide (e.g., anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

Kits containing the agents and instructions necessary to perform the in vitro and in vivo methods as described herein also are claimed. Accordingly, the disclosure provides kits for performing these methods which may include non-naturally occurring peptide and/or other composition of this disclosure as well as instructions for carrying out the methods of this disclosure such as collecting tissue and/or performing the screen, and/or analyzing the results, and/or administration of an effective amount of an anticancer agent. These can be used alone or in combination with other suitable anticancer agents.

The SUPR peptide sequence was derived as described in WO 2013/033636.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### In Vivo Mouse Studies

40 NCr homozygous athymic (nude) mice (six to eight weeks-old) were purchased from the National Cancer Institute. An aliquot of 2×10⁶ SKBR-3 cells were suspended in 200 ml of PBS and injected subdermally in the right thigh of each animals. Treatment began 7 days after inoculation of a non-naturally occurring peptide having SEQ ID NO:1 at a total peptide concentration of 7 mgs/Kg 3× a week by IV injection. Tumor growth was monitored weekly for four weeks. Tumor volume measurements were made by following standard protocols using an electronic caliper.

### Example 2

### Synthesis of Cyclized SUPR Peptides

All solvents were purchased from Sigma. All other peptides were synthesized using fmoc-Gly-Wang resin (250 mgs, 0.15 mmoles) unless otherwise specified. Standard couplings were carried out with 5 eq. of monomer on a PS-3 automated peptide synthesizer (Protein Technologies). Fmoc deprotection was carried out with 20% methyl piperidine at room temperature for 10 min. After the addition of the N-terminal amino acid, peptides were capped with glutaric anhydride. Following N-terminal capping, lysine(mmt) was selectively deprotected with 3% DCM, 1.5% TIS, and 1.5% EDT in DCM for 1 hr at RT. After washing resin with NMP, cyclization on resin was accomplished by the addition of HATU (5eq) and DIEA (10eq) and rotating for 1 hr at RT. Following cyclization, deprotection, cleavage with 95% TFA, filtration and ether extraction, the crude product was purified on a Vydac C-18 reverse phase column using gradient elution (0% B for 5 min, 10-50% B in 40 min. Solvent A: H20 with 0.1% TFA, Solvent B: CH3CN with 0.035% TFA. Lyophilized solid was reconstituted in DMSO and quantitated by absorbance at 280 nm (ε280=9970 L mol-1 cm-1). Yield=10-25%.

### Example 3

### Cell Culture of BT474 (HER-2+) Cells

BT474 cells were purchased from ATCC (Manassas, Va.). BT474 ells were cultured in DMEM (ATCC, Manassas, Va.) supplemented with 10% FBS (Thermo Scientific, Grand Island, N.Y.) at 37° C. and 5% CO₂ following standard conditions. Cells were plated in 96-well black walled, clear bottom plates (Sigma, St. Louis, Mo.) at 2500-100000 cells/well. Indicated amounts of peptide were added with 2%, or less, DMSO to cells and incubated overnight. Proliferation was measured using a BrdU Cell Proliferation Assay Kit (Cell Signaling Technology, Danvers, Mass.). The BrdU compound was given to the cells for 2 hr. Following the manufacturer's protocol, cells were analyzed by UV absorbance measurements at 450 nm. Peptide signal was normalized to cells incubated with 2% DMSO but no peptide. IC₅₀ data was generated by fitting the data to a drug response equation (Log(drug) vs. Response, GraphPad Prism 5.0).

### Example 4

### Affinity Measurements of SUPR Peptide

FACS analysis on BT474 (HER-2+) cells was used to determine affinity. Briefly. BT474 cells were incubated with 0, 10 nM, 20 nM, 30 nM, or 40 nM SUPR-4m-Cy5 at 4° C. for 30 min followed by FACS analysis. The mean fluorescence for each concentration was determined (triplicate samples) and plotted as a function of probe concentration.

The results are shown in FIG. 3. The error bars represent the standard deviation. The dissociation constant (KD) was obtained by nonlinear regression of the data in Graphpad. The analysis shows a 7.2 nM binding affinity, close to the reported 2 nM affinity for the antibody pertuzumab.

### Example 5

### Determination of Optimal Dosing for Her2 SUPR Peptide

Preliminary optical imaging experiments were carried out with both SUPR-2m-Cy5 and SUPR-4m-Cy5 in a subcutaneous SKOV3 model of Her2-positive cancer. Briefly, 10 nmol of SUPR-2m-Cy5 or 50 nmol SUPR-4m-Cy5 was injected via the tail vein of mice, and images were acquired on the IVIS Lumina scanner at 1 hr-4 hr and after 24 hr in the case of SUPR-2m-Cy5.

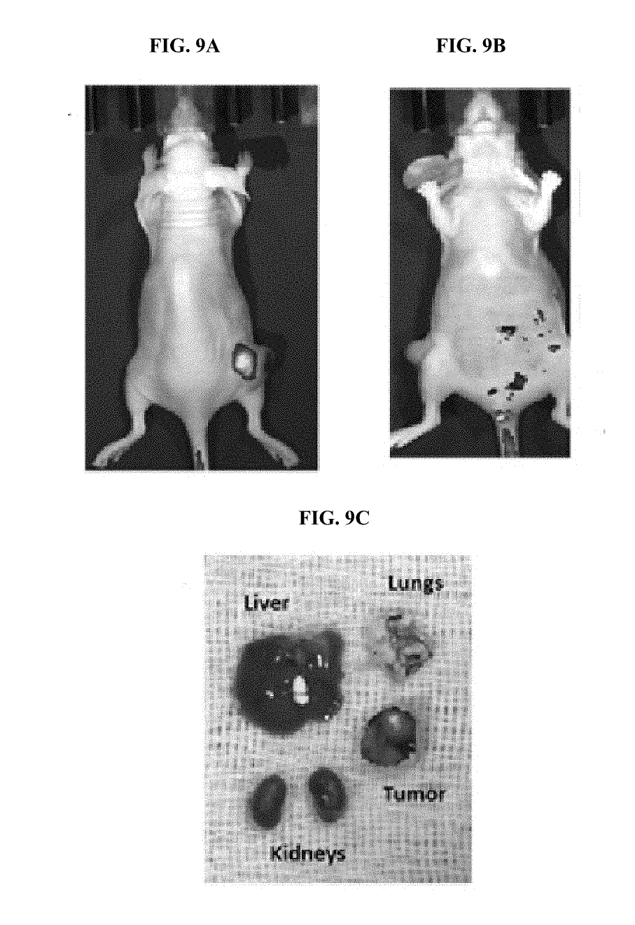

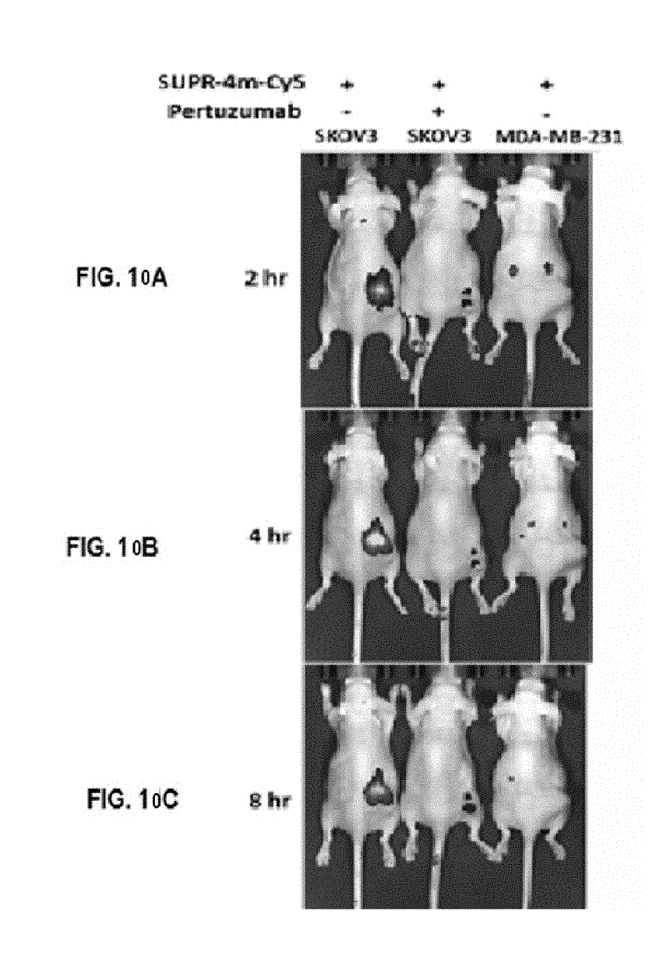

As seen in FIG. 9A (dorsal view of mouse) and FIG. 9B (ventral view of mouse), SUPR-4m-Cy5 showed excellent tumor uptake within 3 hr of injection. The fluorescent signal was concentrated in the non-necrotic region of the tumor (FIG. 9A), and very little background signal is observed in either the dorsal or ventral views. Post-mortem necropsy was performed and the isolated tissues visualized in the same manner. As seen in FIG. 9C, the tumor showed excellent probe uptake with almost no signal observed in the liver, lungs, or kidneys.

### Example 6

### Blocking SUPR Peptide Labeling with Antibody and Determination of Biodistribution

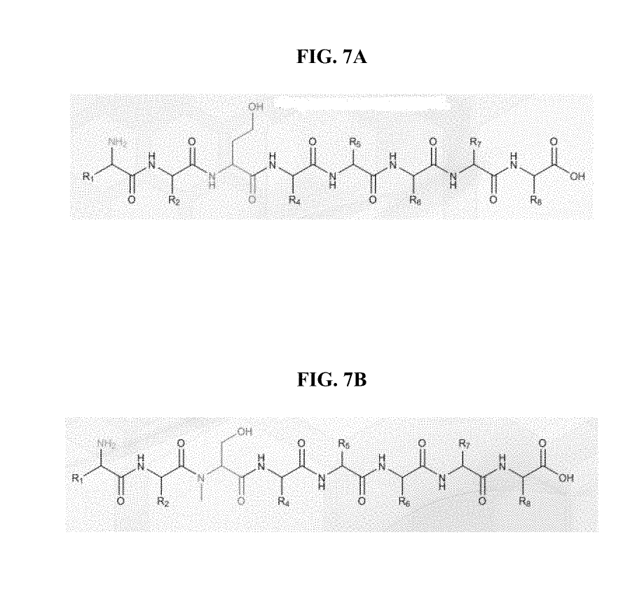

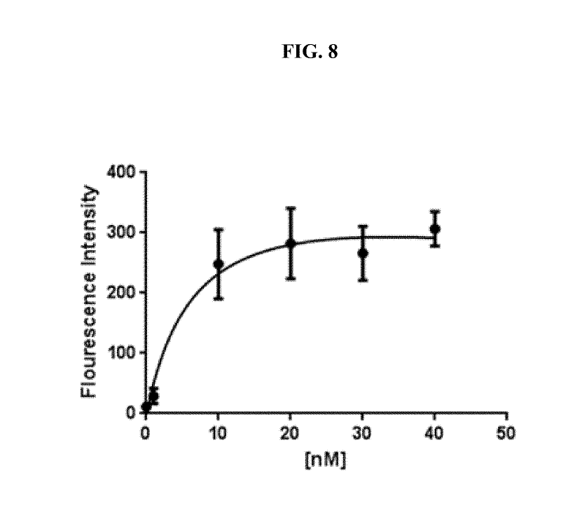

HER-2⁺(SKOV3) and HER-2⁻(MDA-MB-231) subcutaneous flank tumors were employed to determine the in vivo selectivity of the SUPR-4m-Cy5 optical probe at 10 nmol injected dose. As seen in FIG. 7, tumor uptake is observed within 2 hr in the SKOV3 model. Pre-injection 16 hr prior with Pertuzumab (100 mg/kg) significantly reduced this uptake.

The MDA-MB-231 tumor showed >3-fold lower uptake than SKOV3 tumor, indicating that uptake was dependent on expression of unoccupied Her2 receptor. Signal is seen in the kidneys at 2 hr and 4 hr post-injection, particularly in the MDA-MB-231 model, suggesting renal clearance. Dorsal imaging showed significant fluorescence in the GI tract within 2 hr, which may be attributed to residual chlorophyll in the feces, a well-documented phenomenon when alfalfa-based feeds are used.

### EQUIVALENTS

Those skilled in the art will recognize, or be able to ascertain, using no more than routine experimentation, numerous equivalents to the specific composition and procedures described herein. Such equivalents are considered to be within the scope of this disclosure, and are covered by the following claims.

What is claimed is:

1. A Scanning Unnatural Protease Resistant (SUPR) peptide comprising a non-naturally occurring amino acid sequence of MVCVVLYDDK.

2. The peptide of claim 1, wherein the M at position 1 is substituted with norvaline, norleucine, or alanine.

3. The peptide of claim 1, wherein the Vat position 2 is substituted with I or L.

4. The peptide of claim 1, wherein the V at position 4 is substituted with Y, F, P, D, E, or M.

5. The peptide of claim 1, wherein the V at position 5 is substituted with Y, F, D, E, W, C, G, or P.

6. The peptide of claim 1, wherein the L at position 6 is substituted with Y, F, V, V, I, P, or C.

7. The peptide of claim 1, wherein the Y at position 7 is substituted with V, E, or D.

8. The peptide of claim 1, wherein the D at position 8 is substituted with S, T, E, Y, F, A, P, or V.

9. The peptide of claim 1, wherein the D at position 9 is substituted with E, G, L, I, or V.

10. The peptide of claim 1, wherein the K at position 10 is substituted with lysine derivatives e.g. Orn.

11. A peptide, having the structure:

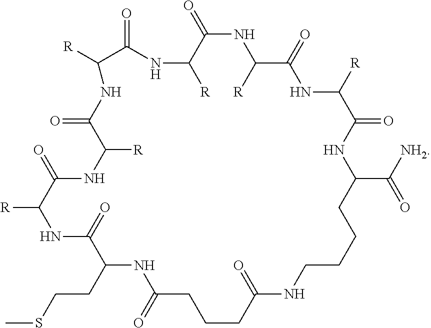

12. A peptide, having the structure:

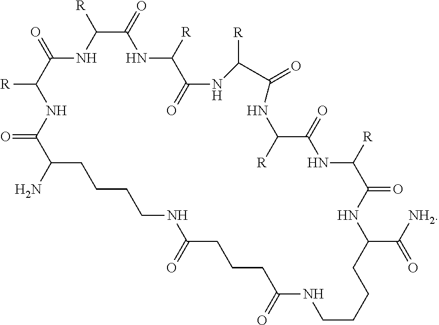

13. A peptide, having the structure:

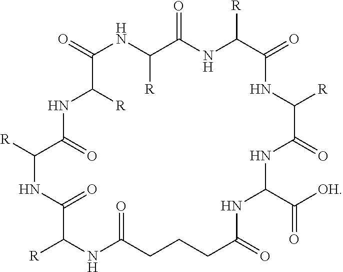

14. A peptide, having the structure:

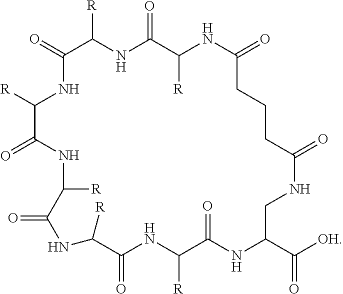

15. A peptide, having the structure:

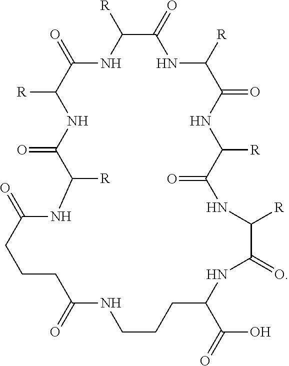

16. A peptide, having the structure:

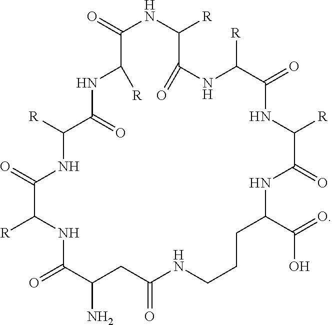

17. A peptide, having the structure:

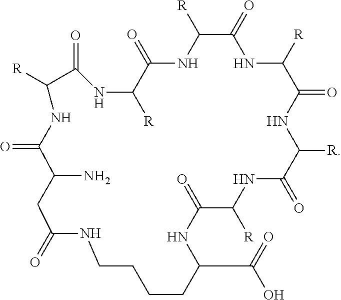

18. A peptide, having the structure:

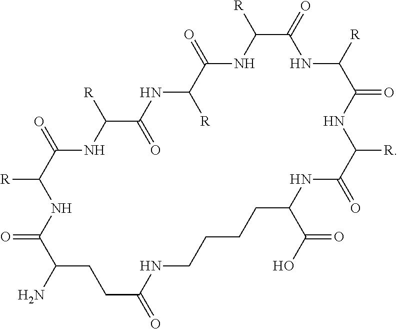

19. A peptide, having the structure:

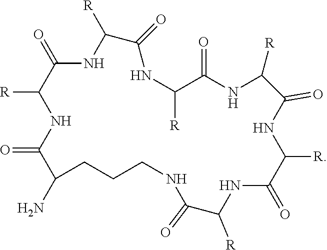

20. A peptide, having the structure:

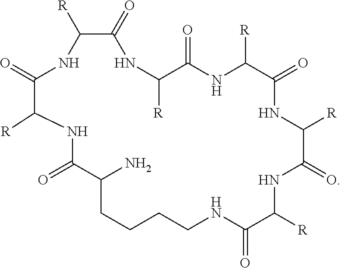

21. A peptide, having the structure:

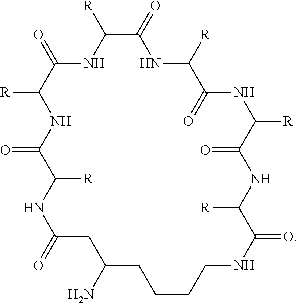

22. A peptide, having the structure:

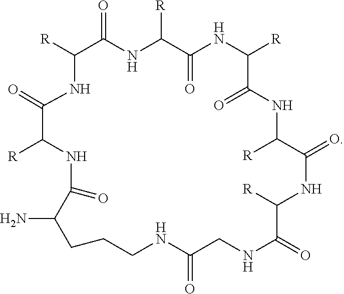

23. A peptide, having the structure:

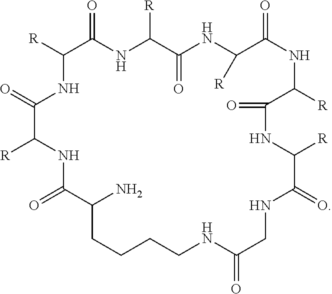

24. A peptide, having the structure:

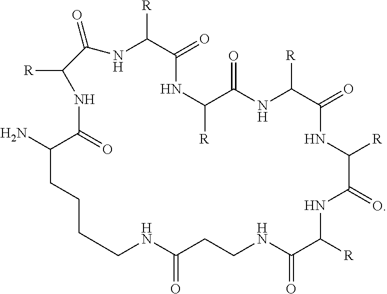

25. A peptide, having the structure:

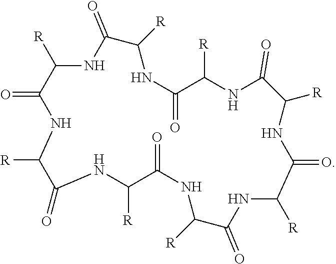

26. A peptide, having the structure:

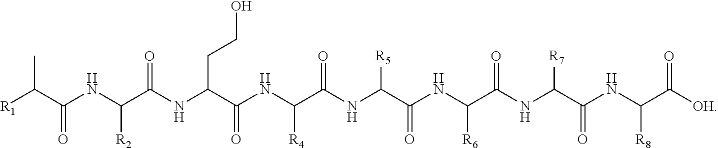

27. A peptide, having the structure:

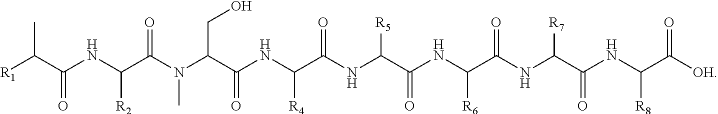

28. The peptide of claim 1, comprising a label.

29. The peptide of claim 28, wherein the label is a dye.

30. The peptide of claim 29, wherein the dye is Cy5.

31. The peptide of claim 28, wherein the label is an in vivo imaging agent.

32. The peptide of claim 31, wherein the in vivo imaging agent is 18F.

33. The peptide of claim 1, wherein the label is a cytotoxin or radioactive atom such as 90Y that can be used for therapeutic applications.

34. The peptide of claim 1, further comprising a pharmaceutically acceptable carrier.

35. A method for inhibiting the growth of an HER-2+ breast cancer cell, comprising contacting the cell with a therapeutically effective amount of the peptide of claim 1.

36. A method for treating HER-2+ breast cancer in subject in need thereof, comprising administering to the subject the therapeutically effective amount of the peptide of claim 1.

37. A method imaging a HER-2+ cell in a subject, comprising administering to the subject the HER-2-specific cyclized SUPR peptide of claim 28 in an amount sufficient to image the cell.

38. A kit for one or more of: inhibiting the growth of a HER-2+ breast cancer cell, for treating a HER-2+ breast cancer, or for imaging a HER-2+ cancer in a subject, comprising the peptide of claim 1 and instructions for use.

39. The kit of claim 38, further comprising a label.


SEQUENCE LISTING (1 sequence)

SEQ ID NO: 1 - protein, 10 aa, Artificial Sequence
  Synthetic Peptide
MVCVVLYDDK